(19) United States
(12) Patent Application Publication
(10) Pub. No.: US 2008/0032308 A1
(43) Pub. Date: Feb. 7, 2008

(54) ISOLATING, POSITIONING, AND SEQUENCING SINGLE MOLECULES

(76) Inventors: Xing Su, Cupertino, CA (US); Narayanan Suadararajan, San Francisco, CA (US); Tae-Woong Koo, Cupatino, CA (US)

Correspondence Address: INTEL/BLAKELY, 1279 OAKMEAD PARKWAY, SUNNYVALE, CA, 94085-4040, US

(21) Appl. No.: 11/845,698

(22) Filed: Aug. 27, 2007

Related U.S. Application Data
(62) Division of application No. 10/897,190, filed on Jul. 21, 2004, now abandoned.
Application No. 10/897,190 is a continuation-in-part of application No. 10/781,238, filed on Feb. 18, 2004, now abandoned.

(51) Int. Cl. C12Q 1/68

(52) U.S. Cl. 435/6

(57) ABSTRACT

Devices and methods for isolating, detecting, and positioning single polymeric molecules without the need for expensive equipment are provided. The disclosed devices and methods allow for a molecule to be quickly and efficiently transported to a specific sub-micron area. Such devices are useful, for instance, for performing analyses in which the sequence of a polymer of interest is determined.

### CROSS-REFERENCE TO RELATED APPLICATIONS

The present application is a continuation-in-part of U.S. patent application Ser. No. 10/781,238, filed Feb. 18, 2004, now pending, the disclosure of which is considered a part of and is incorporated by reference in the present application.

### TECHNICAL FIELD

Embodiments of the present invention relate generally to molecular detection, immobilization, isolation, positioning, and identification.

### BACKGROUND

The sensitive and accurate detection, isolation, and identification of single molecules from biological and other samples has widespread application in medical diagnostics, pathology, toxicology, environmental sampling, chemical analysis, forensics and numerous other fields. To date, however, dependable methods of single molecule detection have proven to be an elusive goal. One problem in being able to detect and isolate a small object such as a single molecule is that as the object to be detected gets smaller, it becomes harder to distinguish from the medium surrounding it. In instances where fluorescent molecular labels have been used to aid detection in solution, the single fluorescent molecule must be distinguishable from the background associated with the solution. For single molecule detection, the smallest possible sample volumes are used because the signal from a single molecule is independent of the sample volume. However, the background is always proportional to the sample volume and therefore single molecule detections are based upon the use of sample volumes of 10 μL or less in order to minimize the background contribution.

Because of this small volume limitation, methods for isolating and positioning single molecules, such as fluorescently labeled DNA fragments, for further analysis, have relied on methods such as hydrodynamic focusing to attain sample volumes of about 1 to about 10 pL. In hydrodynamic focusing, a sample stream is introduced into a rapidly flowing sheath stream from a small orifice. The focused sample stream is then crossed with a tightly focused excitation laser beam having a diameter of from about 10 μm to less than 1 μm. The emitted light is collected by imaging detection optics such as a high numerical aperture microscope objective, passed through a spatial filter or slit, and imaged onto a sensitive detector. (See Ambrose et al. Chem. Rev. 99: 2929-2956 (1999)).

### BRIEF DESCRIPTION OF THE DRAWINGS

In order that the disclosed methods and devices may be better understood, several embodiments will now be described by way of example only and with reference to the accompanying drawings in which,

FIGS. 1A and 1B depict single molecule supports in accordance with this disclosure;

FIGS. 2A-2D depict the immobilization of a single polymer molecule on a support surface such as on a slide, a fiber optic tip, or a microchannel in accordance with this disclosure;

FIG. 3 contains digital photographs of streptavidin-coated beads attached to single DNA molecules that are immobilized within microchannels in accordance with this disclosure;

FIG. 4 depicts how a molecular carrier device interacts with a microfluidic single molecule polymer sequencing system in accordance with this disclosure. Please note that the figures are not to scale.

### DETAILED DESCRIPTION OF THE INVENTION

In one embodiment, the present invention provides devices characterized by a solid support having one or more areas in which a selected number of polymer molecules of interest have been attached. In order that the attachment area contain a selected number of polymer molecules of interest, the attachment area is comprised of regions in which the polymer molecule does not bind. Typically, a target polymer molecule is modified to contain a binding site capable of interacting with a complementary binding site in the attachment area.

The present invention additionally provides methods for creating a solid support characterized by having one or more areas in which a selected number of polymer molecules of interest have been attached. Such methods include, creating an attachment area comprised of binding agents and non-binding agents and attaching a target polymer molecule to a binding agent. The relative density of binding versus non-binding agents is readily manipulated so that a particular number of polymers of interest are attached in a particular attachment area. The attachment of a target polymer within the attachment area can be visualized or otherwise verified. Visualization and verification techniques allow for the selection of attachment areas containing a selected number of target polymer molecules.

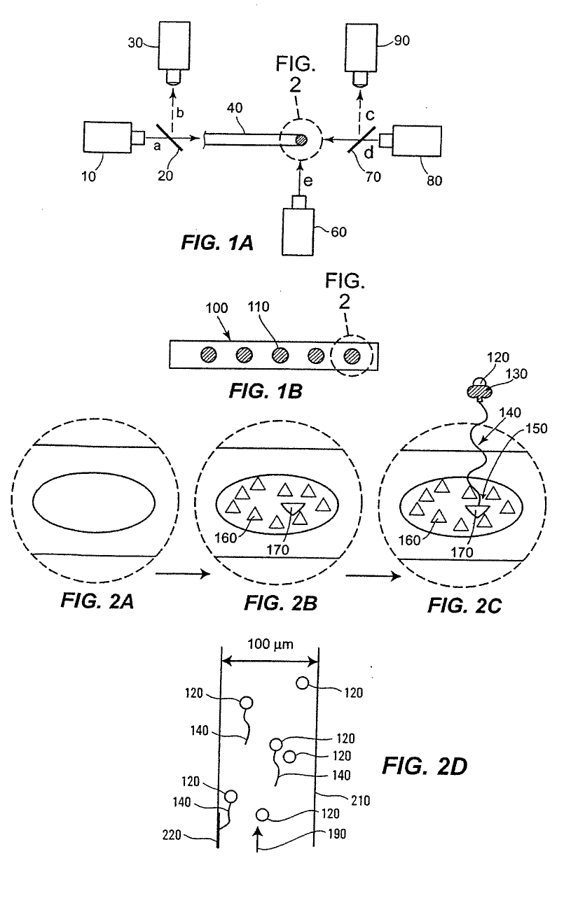

Referring to FIGS. 1A-1B and 2A-2D, polymer molecules 140 are characterized by a covalent molecular arrangement of monomers. Examples of such polymer molecules include, but are not limited to, nucleic acids such as DNA and RNA, proteins, peptides, carbohydrates and other oligosaccharides, plastics, resins, and the like. For ease of illustration, nucleic acids will be used to exemplify the disclosed methods and devices; however, the disclosed methods and devices are not limited to this example.

Virtually any naturally occurring nucleic acid may be prepared and manipulated by the disclosed methods including, without limit, chromosomal, mitochondrial or chloroplast DNA or ribosomal, transfer, heterogeneous nuclear or messenger RNA. Nucleic acids may be obtained from either prokaryotic or eukaryotic sources by standard methods known in the art. RNA can be converted into DNA through the use of a reverse transcriptase enzyme. Methods for preparing and isolating various forms of nucleic acids are known. (See e.g., Berger and Kimmel eds., Guide to Molecular Cloning Techniques, Academic Press, New York, N.Y., 1987; Sambrook, Fritsch and Maniatis, eds., Molecular Cloning: A Laboratory Manual, 2nd Ed., Cold Spring Harbor Press, Cold Spring Harbor, N.Y., 1989). However, embodiments of the present invention are not limited to a particular method for the preparation of target nucleic acids.

Modifications 130 and 150 to the polymer molecule 140, as shown in FIG. 2C, may include any chemical functional group interchange as well as standard molecular labeling techniques. The particular type of modification is chosen to maximize its binding potential with the specific binding molecule and minimize its potential for binding to the functional non-binding molecule or the surface of the support material used in the disclosed methods and devices. Examples of such modifications include, but are not limited to, small functional group changes, such as thiol-modified polymers, amino-modified polymers, aldehyde-modified polymers, carboxy-modified polymers, and the like. Polymers can also be modified with labels or tags that are commonly used in the art. For nucleic acids such labels include, but are not limited to, biotin, fluorescein, digoxigenin, and the like. Such modifications are well known in the art and commercial nucleic acid synthesis vendors provide such modification services (for example Qiagen-operon, Valencia, Calif.).

A linear polymer to be immobilized can be modified with either the same (symmetric modification) or different (asymmetric modification) chemical modifications at each of its two ends. For example a particular polymer molecule can be modified with a thiol group at both ends or with a thiol group at one end and a biotin group on the other end. Asymmetric modification allows the polymer molecule to be attached at one end through a particular type of attachment, for example, a thiol group/gold interaction, leaving the other end free for other manipulations, such as labeling with biotin such that it is available to bind streptavidin, avidin, or a streptavidin or avidin modified substrate.

A specific binding molecule or specific binding agent 170 (see FIGS. 2B and 2C) is a molecule or atom that can form a strong interaction with the polymeric modification. For example, gold forms a covalent binding interaction with thiol-modified polymer molecules; antibodies are available which selectively bind such molecular labels as fluorescein and digoxigenin, and avidin and streptavidin have a non-covalent binding interaction with biotin with an energy equivalent to some covalent bonds. Specific binding molecules 170 include chemical modifications of a substrate surface with small functional groups which can specifically bind to the chemical modification on the polymer. For example, aldehyde modified surfaces easily attach to amino group modified polymer molecules. The property of this latter interaction has resulted in a variety of commercially available biotin labeled or tagged molecules that can be used to immobilize a molecule on a solid support which is functionalized with avidin or streptavidin molecules. The term antibody as used herein includes polyclonal and monoclonal antibodies as well as fragments thereof, recombinant antibodies, chemically modified antibodies and humanized antibodies, all of which can be single-chain or multiple-chain.

A functional non-binding molecule or a functional non-binding agent 160 (see FIGS. 2B and 2C) is a molecule or atom which does not form a strong interaction with the polymeric modification. For example, platinum (Pt), and copper (Cu) do not have a binding interaction with thiol groups; a carboxy-modified substrate will not bind to thiol modified polymers; bovine serum albumin (BSA) and bovine IgG (BIgG) do not have a binding interaction with biotin; and streptavidin or avidin do not bind to digoxigenin.

In one embodiment, the specific binding molecule 170 and the functional non-binding agent 160 used are approximately the same size and molecular weight. For example, Au (MW 197) is of a similar size and molecular weight as Pt (195), but not Ag (MW 107.9) or Cu (65.5). Likewise, BSA (MW 65 kD) is of a similar size and molecular weight as avidin (MW 66 kD).

The microarea 110 (FIG. 1B) can be of any particular size. In one embodiment, at least one of the dimensional distances (e.g. diameter, height, width, etc.) of the microarea 110 is at least two times the length of the polymer molecule to prevent the polymer molecule 140 from attaching at both modified termini. For example, for a DNA molecule comprising about 50,000 base pairs, this distance is about 17 microns long. Therefore, when such a DNA molecule is used this microarea 110 can range from about 17 microns to about 70 millimeters.

In one embodiment of the invention, the specific binding molecule 170 is mixed with an effective molar amount of the functional non-binding molecule 160 such than only one modified polymer molecule 130-150 can be immobilized in a given microarea 110 on a solid support 40 or 100 (FIGS. 1A and 1B). The molar ratio of specific binding molecule 170 to functional non-binding molecule 160 (the substrate ratio) can be changed and experimentally verified depending on the desired distance between the molecules to be immobilized. Any substrate ratio can be used. Ratios of the specific binding molecule to the functional non-binding molecule may range from about 1:10¹⁰ to about 10:1 depending on the particular combination of specific binding molecule and functional non-binding molecule. For example, if gold is the specific binding molecule and copper is the functional non-binding molecule, then a ratio of about 1:10⁸ respectively is may be used. If monomeric avidin is the specific binding molecule and BSA is the functional non-binding molecule, then a ratio of about 1:10⁷ may be used.

The molar ratio of modified polymer to specific binding molecule 170 to functional non-binding molecule 160 (the target ratio) can also be changed and experimentally verified depending on the desired distance between the molecules to be immobilized. Any target ratio can be used. Target ratios may range from about 1:10¹⁰ to about 1:0 depending on the particular combination of specific binding molecule and modified polymer. For example, if monomeric avidin is the specific binding molecule and the polymer is modified with streptavidin, then a ratio ranging from about 1:10 to about 1:1000 is advantageous.

In one embodiment, one may use a formulation containing only specific binding molecule and no functional non-binding molecule. In this embodiment if a symmetrically modified polymer is used, most of the polymer molecules will be attached to the substrate at both ends and only a few polymer molecules will be immobilized with a free terminus. Because polymer molecules with free termini are limited in this embodiment they will have a lower density, however they are still easily detected and isolated. Polymer molecules with no free ends do not interfere with the isolation of polymer molecules with free ends.

In one embodiment, the specific binding molecule 170 used may have multiple binding sites. For example, normal avidin and streptavidin have about 4 binding sites in each molecule. In this embodiment a suitable amount of a blocking molecule may be added such that there is only one effective binding site per specific binding molecule. For example if avidin is used, free biotin can be mixed with the biotin-modified polymer in about a 3:1 ratio such that 3 of the 4 binding sites are blocked from binding the modified polymer. An effective binding site density can be calculated from the density of total binding sites multiplied by the ratio of blocking molecules to target molecules, assuming the total number of blocking molecules and target molecules is far greater than the total number of binding sites.

Various types of solid supports 40, 100 (FIGS. 1A and 1B) can be used in the disclosed methods and devices. Examples of suitable solid supports include, but are not limited to, plates, slides, films, strips, rods, tubes, beads, and the like. These supports can be made from a variety of materials including, but not limited to, metal, glass or other silica-based materials, polymeric resin-based materials, and the like. For ease of illustration, a metal or glass slide 100 and an optical fiber 40, as shown in FIGS. 1A and 1B, will be used to exemplify the disclosed methods and devices, however, the disclosed methods and devices are not limited to these examples.

Still referring to FIGS. 1A-1B and 2A-2C, the specific-binding 170 and functional non-binding agents 160 are attached to the solid support 40 or 100 by a variety of methods known in the art depending on the support material and the molecules to be used. For example, if the support is metal, and gold and silver are the specific binding molecule and the functional non-binding molecule, respectively, standard metal annealing methods may be used. If the support material is glass, and avidin and BSA are the specific binding molecule and the functional non-binding molecule, respectively, standard covalent coupling methods may be used.

Standard covalent coupling methods comprise providing a reactive group either to the molecule to be attached to the surface or to the surface itself. Examples of these reactive groups include, but are not limited to, carboxyl, amino, hydroxyl, hydrazide, amide, chloromethyl, aldehyde, epoxy, tosyl, thiol, and the like, which are commonly used in the art.

For example, aldehyde modified glass surfaces have been shown to be especially suitable for the present application for creating protein-coated surfaces. The existence of terminal amino groups on the proteins used as functional-nonbinding and functional binding compounds in the disclosed methods and devices ensures their availability for complementary attachment to one or more aldehyde groups on the surface of the support. After reducing the imine produced this group has proven to be very stable over time. Additionally, the chemistry involved in attaching ligands to either of these groups has been widely explored and the reagents involved are readily commercially available.

Aldehyde-modified glass surfaces can be prepared by at least two processes. The first process involves immersing a polished and NoChromix and Piranha cleaned surface for 30 minutes in a hydrolysed solution of 0.5% glycidyloxypropyltrimethyoxysilane (GPTMS), 4.5% ethyltrimethoxysilane (ETMS) in 50 mM pH 5.7 4-morpholineethanesulfonic acid (MES), followed by a solution of 1 mM sodium periodate (NaIO₄) in pH 7.2 PBS for 1 hr at room temperature (RT). The second process involves sonicating the polished and cleaned surfaces in 2% GPTMS in 95% EtOH/5% deionized water (DI H₂O) for 2 minutes, rinsing with ethanol (EtOH) and drying, and then immersing the surfaces in a solution of 1 mM NaIO₄ in pH 7.2 PBS for 1 hr at room temperature.

The microarea(s) 110 to be coated with the specific binding molecule 170 and non-functional binding molecule 160 mixture may be coated by standard inkjet printing, standard photolithography, contact printing techniques or techniques for microarray fabrication to deposit the specific binding and non-functional binding molecules in given areas on the surface of the support 40, 110. The support 40, 110 can be coated in multiple positions.

In certain embodiments of the disclosed methods and devices, specific areas of the support 40, 110 can be precoated with protecting groups so that these areas cannot be coated with the mixture of the specific binding molecule and the functional non-binding molecule. The specific protecting groups used depend on the type of surface to be protected. Examples of protecting groups for glass substrates include, but are not limited to, substituted and unsubstituted alkyl ethers, substituted and unsubstituted benzyl ethers, silyl ethers, esters, carbonates, sulfonates, and the like. (See e.g., T. W. Greene, Protective Groups in Organic Synthesis. Wiley & Sons. (1991)). Removing these protecting groups, either chemically, or mechanically by cleaving or etching the support surface, exposes a fresh substrate surface which can be coated with the mixture of the specific binding molecule 170 and the functional non-binding molecule 160.

The modified polymer molecule shown at 130, 140, 150 in FIG. 2C can then be immobilized on coated microareas 110 of the support 40 or 100 by contacting the modified polymer molecule with the coated solid support. For example if the substrate is coated with gold, then the thiol-modified polymer is applied over the gold patch allowing the formation of covalent attachment between gold surface and thiol group. Any unbound polymer molecules can be removed by washing the coated area with a buffer solution.

In other embodiments the polymer can be synthesized on the substrate. Using nucleic acids as an example, a polydeoxyadenosine (poly (dA)) primer modified with a thiol group on one end can be first immobilized on a surface using the above methods. Then a template DNA molecule with a polydeoxythymidine (poly (dT)) sequence (either labeled or unlabeled) is allowed to hybridize or anneal to the preimmuobilized poly(dA) through adenosine-thymidine hybridization. The poly (dA) sequence can then be extended by a DNA polymerase in the presence of nucleotides and other required reagents. Unused primer molecules can then be separated from the desired, immobilized nucleic acid molecule.

The detection of a single bound polymer molecule 140 and the verification of the spacing between individual bound polymer molecules can be accomplished by a variety of methods depending on the modification at the free terminus of the polymer molecule 130. These methods include, but are not limited to, labeling the immobilized polymer molecule by contacting it with a fluorescently labeled specific binding molecule or other label 120 that is specific for the modification on the polymer's free terminus. For example, if a nucleic acid molecule is modified with biotin at its free terminus, the immobilized nucleic acid can be labeled with avidin-tagged or labeled with fluorescent molecules or with a streptavidin bead. Alternatively the polymer molecule can be detected by contacting the immobilized polymer with a fluorescent dye, label, or stain and detecting the individual polymer molecules and scanning the support for fluorescent emission from the label using a single-photon counting device or some other optical detecting device. Likewise, the nucleic acid molecule can be stained by a nucleic acid specific dye, such as, ethidium bromide.

The embodiments of the disclosed methods and devices are not limited by the type or arrangement of detection unit used, and any known detection unit may be used in the disclosed methods and device. If the labels are fluorescent, standard light sources 10, 60, or 80, such as those shown in FIG. 1A, can be used to provide the desired absorption wavelength of common fluorescent dye molecules. Examples of such light sources include, but are not limited to, lasers, mercury or xenon gas lamps (Oriel Instruments) and filters (Omega Optical or Chroma). For example, the tip of an optical fiber 40 is used as the support, such light a can be delivered to the molecule through the optical fiber to which the molecule is attached. In such an embodiment, part of the emitted fluorescent light b is captured by the same optical fiber, and travels back to the other end of the optical fiber. A dichroic mirror 20 can be used as part of this detection method to separate beams or waves of excitation light and emitted fluorescence light, by reflecting the back-scattered fluorescent light toward a detector 30. If the fluorescence from the optical fiber interferes with the fluorescence from the attached molecule, or if a collinear geometry is difficult to implement due to the alignment or the size of the instrument, a forward or side scattering geometry c can be used. In a forward-scattering geometry, excitation light d is delivered to the molecule and part of the emitted fluorescent light b is captured by the optical fiber and travels to detector 30 either directly or reflected by dichroic mirror 20. In a side-scattering geometry, excitation light e is delivered to the molecule and part of the emitted fluorescent light b is captured by the optical fiber and travels to detector 3 either directly or reflected by dichroic mirror 20.

Still referring to FIG. 1A, the optical detector 30 or 90 can be any standard optical detector or array of detectors including, but not limited to, photodiode detectors, avalanche photodiode detectors, Charge-Coupled Devices (CCD) arrays of detectors, Complementary Metal-Oxide Semiconductor (CMOS) arrays, intensified CCD cameras, or any other optical detector with reasonable sensitivity and speed.

CMOS arrays using both N-type and P-type transistors may also be used to realize logic functions. CMOS technology has advantages in that little to no static power dissipation when compared to Negative-Channel Metal-Oxide Semiconductor (NMOS) or bipolar circuitry. Power is only dissipated in case the circuit actually switches. This allows integration of many more CMOS gates on an integrated circuit than in NMOS or bipolar technology, resulting in much better performance.

In order to further reduce the fluorescence generated by the optical fiber, the excitation beam can impinge the attached molecule at an angle outside the collection angle of the optical fiber.

Typically about 4% of the impinging light is reflected from the surface, which is considered as a loss in transmission. In another embodiment, the attachment end of the optical fiber can be coated with dielectric materials designed to allow the fluorescence from the attached molecule to enter the optical fiber with low light loss, while reflecting the excitation light and preventing it from entering the optical fiber. A typical dielectric coating can block the excitation light by factor of 10⁶ and transmit more than 96% of the fluorescence light impinging on the coating.

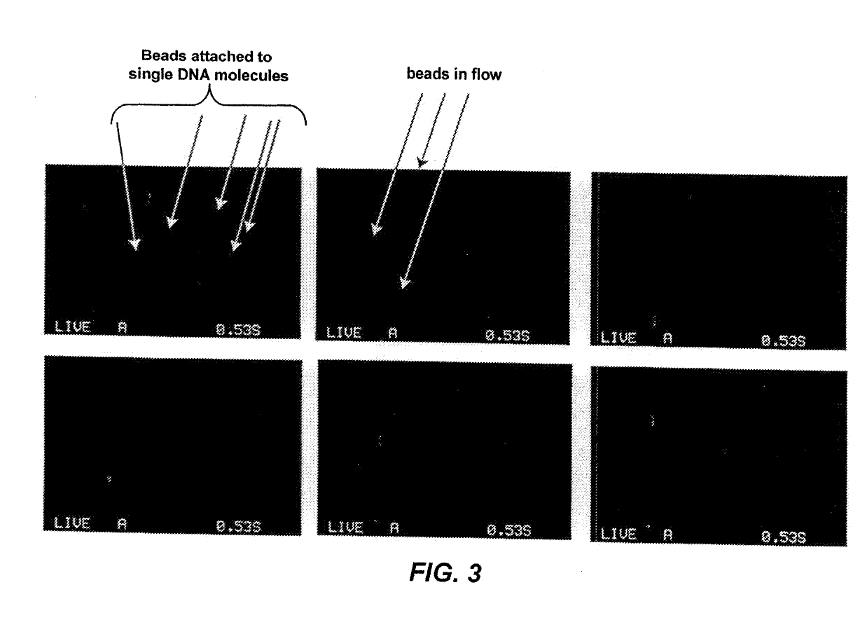

In another embodiment illustrated in FIGS. 2D and 3, the label 120 is a bead and the molecule 140 may detected visually using a microscope or other optically magnifying device. For example, FIG. 3 shows digital photographs of streptavidin-coated beads attached to single DNA molecules that are immobilized within microchannels 210 as also shown in FIG. 2D. In FIG. 2D, beads 120 attached to a single DNA molecule 140 which is also attached to the substrate 220 (large spots) can be differentiated from beads 120 attached to single molecules 140 and unattached beads 120 in the flow 190 After identifying areas where single polymer molecules are attached to the substrate, positions that have single polymer molecules may be marked using microscopy stages and saved for later use.

Sequencing

In a further embodiment, the present invention provides methods for determining the sequence of a target nucleic acid molecule of interest. The sequence of a target nucleic acid molecule can be determined by placing an attachment area containing a target nucleic acid into a reaction chamber of a microfluidic system. The target nucleic acid is then digested, releasing its component monomers from a free terminus of the polymer molecule. The digested component monomers are detected in a manner that allows the sequence of the target nucleic acid to be reconstructed.

The disclosed methods and devices can be used for sequencing single polymer molecules including nucleic acids such as DNA and RNA. As discussed above, methods for preparing and isolating various forms of nucleic acids are known. RNA can be converted into cDNA through the use of a polymase enzyme, such as reverse transcriptase. As described herein, a single DNA molecule can be attached to an attachment region of a solid support. This solid support can be be inserted into an apparatus that allows the monomers of the polymer to be sequentially digested and detected. The sequential detection of the monomeric units of the polymer, in this case, nucleotides, allows the sequence of the nucleic acid to be reconstructed.

Optionally, some of the monomers of the nucleic acids may be labeled for detection. The label attachment may be covalent or non-covalent. In non-limiting examples, labels may be fluorescent, phosphorescent, luminescent, electroluminescent, chemiluminescent or any bulky group or may exhibit Raman or other spectroscopic characteristics. In certain embodiments, nucleotide precursors may be secondarily labeled with bulky groups after synthesis of a complementary strand but before detection of labeled nucleotides. In some embodiments, nanoparticle labels may be used that generate unique optical signals such as luminescence, surface plasmon resonances, or surface-enhanced Raman scattering signals. Labels that generate Raman signals include, for example, composite organic inorganic nanoclusters (COINs) (as described by commonly assigned U.S. patent application Ser. No. 10/830,422).

Nucleotide precursors covalently attached to a variety of labels, such as fluorescent labels, may be obtained from standard commercial sources (for example, Molecular Probes, Inc., Eugene, Oreg.). Alternatively, labeled nucleotide precursors may be prepared by standard techniques well known in the art. The practice of the present invention is not limited to a particular method that may be chosen for preparing labeled nucleotide precursors.

In various embodiments of the present invention, a nucleotide precursor with an incorporated reactive group and/or hapten may be attached to a secondary label, such as, for example, an antibody-containing label. Any type of detectable label known in the art may be used, such as Raman tags, fluorophores, chromophores, radioisotopes, enzymatic tags, antibodies, chemiluminescent, electroluminescent, affinity labels, etc. One of skill in the art will recognize that these and other known label moieties not mentioned herein can be used in the disclosed methods.

The label moiety to be used may be a fluorophore, such as Alexa 350, Alexa 430, AMCA (7-amino-4-methylcoumarin-3-acetic acid), BODIPY (5,7-dimethyl-4-bora-3a,4a-diaza-s-indacene-3-propionic acid) 630/650, BODIPY 650/665, BODIPY-FL (fluorescein), BODIPY-R6G (6-carboxyrhodamine), BODIPY-TMR (tetramethylrhodamine), BODIPY-TRX (Texas Red-X), Cascade Blue, Cy2 (cyanine-2), Cy3, Cy5,5-carboxyfluorescein, fluorescein, 6-JOE (2′7′-dimethoxy-4′5′-dichloro-6-carboxyfluorescein), Oregon Green 488, Oregon Green 500, Oregon Green 5, Pacific Blue, Rhodamine Green, Rhodamine Red, ROX (6-carboxy-X-rhodamine), TAMRA (,N,N′,N′-tetramethyl-6-carboxyrhodamine), tetramethylrhodamine, and Texas Red. Fluorescent or luminescent labels can be obtained from standard commercial sources, such as Molecular Probes (Eugene, Oreg.).

In some embodiments of the disclosed methods and devices, functional groups, such as labels, may be covalently attached to linkers, such as cross-linking agents, so that interactions between template strand, complementary strand and polymerase may occur without steric hindrance.

Standard molecular biology techniques may be used to accomplish the labeling of the DNA polymers. By using labeled deoxynucleotide triphosphates (dNTPs) as precursors, labeled DNA molecules can be synthesized. Method for synthesizing DNA molecules are described in Sambrook et al., Molecular Cloning: A Laboratory Manual, Cold Spring Harbor Laboratory Press, NY, Vol. 1-3 (1989) and D. Glover, DNA Cloning Volume I: A Practical Approach. IRL Press, Oxford, 1985. These techniques include, but are not limited to, a) random primer methods, b) polymerase chain reaction (PCR) methods, c) strand replacement methods, and d) primer extension methods. The random primer method is based on the work of Feinberg (Anal. Biochem. 132: 6-13 (1983) and 137: 266-267 (1984)). Random primers can be obtained by: a) digesting calf thymus or salmon sperm DNA with DNAase I to generate a large population of single-stranded DNA fragments 6-12 nucleotides in length; b) purchasing random oligonucleotides from commercial sources (e.g. Pharmacia, Roche, International Biotehnologies etc.); or c) synthesizing on an automated DNA synthesizer a population of octamers or 9-mers that contains all four nucleotides in every position. Because of their uniform length and lack of sequence bias, synthetic oligonucleotides are preferred. Random Primer DNA labeling kits are commercially available from Panvera and other companies.

The type of DNA polymerase used depends on the nature of the template: a) RNA-dependent DNA polymerase (reverse transcriptase) is used to copy single-stranded RNA templates into cDNA or; b) the Klenow fragment of E. coli DNA polymerase I is used when the template is single stranded DNA. In both cases, the synthesis of DNA is carried out using one labeled type of dNTP and three unlabeled types of dNTPs as precursors to yield DNA wherein a large proportion of a particular type of nucleotide is labeled. Reverse transcriptase kits are commercially available from Qiagen GmbH (Germany) and other companies.

All of these techniques can be performed in one or two steps, depending on the polymerase used. For Klenow and reverse transcriptases, the labeling and primer extension/chain termination reactions can be combined by lowering the concentration of one of the four dNTPs and adding the same labeled dNTP. For all polymerases, including the widely-used T7 DNA polymerase, these two reactions can be performed sequentially. In the labeling reaction, the primer is extended a short time using limiting concentrations of dNTPs and a single labeled dNTP. In the extension/termination step, the extended primers are further extended in the presence of both dNTPs and ddNTPs, leading to sequence specific chain terminations. The principal advantage of this method is that multiple labels are incorporated into each chain and the density of the labels can be controlled by varying the ratios of labeled dNTPs with unlabeled dNTPs.

The PCR method for amplifying DNA is described in U.S. Pat. Nos. 4,683,195 and 4,683,202 assigned to Hoffman-La Roche Inc. and F. Hoffann-La Roche Ltd. In the PCR method, the resulting product can be labeled with either modified nucleotides or modified oligonucleotide primers. Typically, these labels are fluorescent labels because they allow for direct detection, sensitivity, and multicolor capability. Fluorescently labeled deoxynucleotide triphosphates (dNTPs) and fluorescently end-labeled oligonucleotide primers are commercially available for use in PCR product labeling from Molecular Dynamics. PCR primers labeled fluorescently at the 5′ end can be produced de novo during oligonucleotide synthesis or by using chemistries such as the Fluorescent 5′-Oligolabeling Kit from Amersham Pharmacia Biotech.

Techniques capable of detecting and identifying a labeled nucleotide include, but are not limited to, visible light, ultraviolet and infrared spectroscopy, Raman spectroscopy, nuclear magnetic resonance, positron emission tomography, scanning probe microscopy and other methods known in the art. Methods for determining the sequence of partially labeled nucleic acids are disclosed in copending U.S. patent application Ser. No. 10/782,014.

In certain embodiments, a device useful for sequencing a nucleic acid comprises one or more microfluidic channels, for example, to provide connections to a molecule detector, to a waste port, to a polymer loading port, and/or to the source of reactants for cleaving off individual monomers. All these components may be manufactured in a batch fabrication process, as known in the fields of computer chip manufacture or microcapillary chip manufacture. In some embodiments of the disclosed methods and devices, the sequencing apparatus and its individual components may be manufactured as a single integrated chip. Such a chip may be manufactured by methods known in the art, such as by photolithography and etching. However, the manufacturing method is not limiting and other methods known in the art may be used, such as laser ablation, injection molding, casting, or imprinting techniques. (See e.g., Duffy, D. C. et al., “Rapid Prototyping of Microfluidic Systems in Poly(dimethylsiloxane)” Anal. Chem. 70:4974-4984, (1998).) Methods for manufacture of nanoelectromechanical systems may be used for certain embodiments of the disclosed methods and devices. (See e.g., Craighead, Science 290:32-36, (2000).) Microfabricated chips are commercially available from sources such as Caliper Technologies Inc. (Mountain View, Calif.) and ACLARA BioSciences Inc. (Mountain View, Calif.). The material comprising the sequencing apparatus and its components may be selected to be transparent to electromagnetic radiation at excitation and emission frequencies used for the detection unit. Glass, silicon, and any other materials that are generally transparent in the visible frequency range may be used for construction of the apparatus.

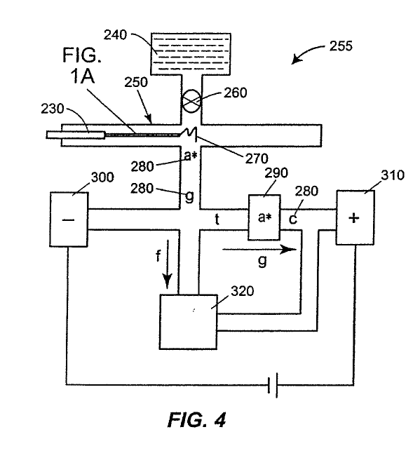

Referring to FIG. 4, in one embodiment, the polymers can be sequenced by placing the molecular carrier in a microfluidic device equipped for single polymer molecule sequencing and detection. The molecular carrier of FIG. 1A is positioned in the system with a positioning device 230 such that a single molecule 270 is positioned in the reaction chamber 250 of the sequencing device 255. The positioning device 230 can be fitted with a seal (not shown) such that the carrier can be moved in and out without causing leakage. Then, using a combination of chemical or enzymatic methods and microfluidics, each monomer (either labeled or non-labeled) from the polymer strand can be sequentially cleaved and transported into a collection volume for detection. For example, if the polymer molecule is a nucleic acid, a buffered enzyme solution 240 with exonuclease activity is then flowed using a flow control device 260 into the reaction chamber 250 of the channel to digest the DNA strand and release the individual labeled or unlabeled nucleotide monomers 280 one at a time. Preferably this enzyme solution is pumped into the reaction chamber 250 at a predetermined rate using the flow control device 260. The cleaved nucleotide monomers 280 are carried/transported in the flow f and g directed through a sample cell 290 where the signal from the monomer or its label is sequentially detected. A electrical field generated by an anode 300 and a cathode 310 may be used to help focus the monomers through the sample cell. The nucleotide monomers may optionally be carried or transported to a collection or waste chamber 320.

Examples of suitable exonucleases, include, but are not limited to exonuclease 1, lambda exonuclease, or a DNA polymerase with exonuclease activity, such as T4 DNA polymerase or T7 DNA polymerase. Exonuclease 1 digests single stranded DNA from the 3′ to 5′ end; lambda exonuclease digests double stranded DNA from the 5′ to 3′ end; and T4 DNA polymerase (exonuclease) and T7 DNA polymerase (exonuclease) digest single and double stranded DNA from the 3′ to 5′ end.

The digested monomers can be detected by a variety of techniques and the embodiments of the disclosed methods and devices are not limited by the type of detection unit used; any known detection unit may be used in the disclosed methods and apparatus. For example, the nucleic acid monomers can be detected and identified using surface enhanced coherent anti-Stokes Raman spectroscopy (SECARS) according to the methods and instrumentation disclosed in copending U.S. application Ser. No. 10/688,680. For Raman detection, an interior surface of the detection cell can be coated with metal, metal nanoparticles, aggregates of metal nanoparticles, or crosslinked metal nanoparticles or aggregates thereof, comprising metals such as silver or gold, for SERS or SECARS signal enhancement. A label may be detected using any detector or detection scheme known in the art, such as a spectrophotometer, luminometer, NMR (nuclear magnetic resonance spectroscopy), mass-spectroscopy, imaging systems, charge coupled device (CCD), CCD camera, photomultiplier tubes, avalanche photodiodes, AFM (atomic force microscopy), or S™ (scanning tunneling microscopy).

Nanopore detection technology may also be used to detect monomers. Nanopores measure the changes in ionic conductivity when a particular type of molecule passes through a it or membrane channel containing nanopores. Nanopore diameters are typically on the order of a few nanometers. The nanopore is filled only in an electrolyte solution and a voltage bias induced by a cathode and anode arrangement causes ions to flow through the nanopore in the sample cell. The ionic current flow is on the order of picoamperes. When single molecules are drawn into the nanopore by the voltage bias, the molecules partially obstruct the nanopore and reduce its ionic conductivity. Quantifying the reduction of the ionic conductivity allows for the direct characterization of a labeled or unlabeled monomer on a nanosecond or microsecond time scale without the need for amplification. The sensitivity of this technique can be increased by covalently tethering a molecule near the pores lumen to act as an additional sensor that can selectively, but reversibly, bind to the different types of molecules to be analyzed. For example, when a molecule that more strongly interacts with the sensor molecule is drawn into the lumen of a nanopore by the voltage bias, it is more likely to have an interaction with the sensor molecule that increases its time in the nanopore and creates a signature time duration of ionic conductivity reduction. Likewise, when a molecule that only weakly interacts with the sensor molecule is drawn into the lumen of a nanopore, its time in the nanopore is not signficantly increased, again creating a signature time duration of ionic conductivity reduction. Plotting the translocation duration vs. the change in ionic conductivity allows for the identification of each unique type of labeled or unlabeled monomer. Examples of such sensor molecules for nucleotide monomers include a binding molecule for the label or a base pair complement to the nucleotide. Nanopores have been used to sequence codons in a single molecule of DNA (See Wang et al. Nature Biotechnology, 19: 622-623 (2001); Meller et al. Proc. Nat'l. Acad. Sci. 97: 1079 (2000)). A labeled nucleotide can have a larger size and different chemical properties compared to normal nucleotides.

In alternate embodiments, labeled nucleotides attached to luminescent labels may be detected using a light source and photodetector, such as a diode-laser illuminator and fiber-optic or phototransistor detector. (See Sepaniak et al., J. Microcol. Separations 1:155-157 (1981); Foret et al., Electrophoresis 7:430-432 (1986); Horokawa et al., J. Chromatog. 463:39-49 (1989); U.S. Pat. No. 5,302,272.) Other exemplary light sources include vertical cavity surface-emitting lasers, edge-emitting lasers, surface emitting lasers and quantum cavity lasers, for example a Continuum Corporation Nd-YAG pumped Ti:Sapphire tunable solid-state laser and a Lambda Physik excimer pumped dye laser. Other exemplary photodetectors include photodiodes, avalanche photodiodes, photomultiplier tubes, multianode photomultiplier tubes, phototransistors, vacuum photodiodes, silicon photodiodes, and charge-coupled devices (CCDs). Using surface-enhanced Raman scattering, fluorescence and other optical methods, single nucleotide molecules can be detected and identified. (see Kneipp et al., Phys. Rev. E, 57: R6281 (1998); Keir et al., Anal. Chem., 74: 1503 (2002); Doering et al., J. Phys. Chem. B, 106: 311 (2002)).

In some embodiments, the photodetector, light source, and nanopore may be fabricated into a semiconductor chip using known N-well Complementary Metal Oxide Semiconductor (CMOS) processes (Orbit Semiconductor, Sunnyvale, Calif.). In alternative embodiments of the disclosed methods and devices, the detector, light source and nanopore may be fabricated in a silicon-on-insulator CMOS process (for example, U.S. Pat. No. 6,117,643). In other embodiments of the disclosed methods and devices, an array of diode-laser illuminators and CCD detectors may be placed on a semiconductor chip (U.S. Pat. Nos. 4,874,492 and 5,061,067; Eggers et al., BioTechniques, 17: 516-524 (1994)).

In certain embodiments, a highly sensitive cooled CCD detector may be used. The cooled CCD detector has a probability of single-photon detection of up to 80%, a high spatial resolution pixel size (5 microns), and sensitivity in the visible through near infrared spectra. (Sheppard, Confocal Microscopy: Basic Principles and System Performance in: Multidimensional Microscopy, Springer-Verlag, New York, N.Y., pp. 1-51 (1994).) In another embodiment of the invention, a coiled image-intensified coupling device (ICCD) may be used as a photodetector that approaches single-photon counting levels (U.S. Pat. No. 6,147,198). A small number of photons triggers an avalanche of electrons that impinge on a phosphor screen, producing an illuminated image. This phosphor image is sensed by a CCD chip region attached to an amplifier through a fiber optic coupler. In some embodiments of the disclosed methods and devices, a CCD detector on a chip may be sensitive to ultraviolet, visible, and/or infrared spectra light (for example as described in, U.S. Pat. No. 5,846,708).

In some embodiments, a nanopore may be operably coupled to a light source and a detector on a semiconductor chip. In certain embodiments of the disclosed methods and devices, the detector may be positioned perpendicular to the light source to minimize background light. The photons generated by excitation of a luminescent label may be collected by a fiber optic. The collected photons are transferred to a CCD detector and the light detected and quantified. Methods of placement of optical fibers on a semiconductor chip in operable contact with a CCD detector are known (for example, as described in U.S. Pat. No. 6,274,320).

In some embodiments, an avalanche photodiode (APD) may be made to detect low light levels. The APD process uses photodiode arrays for electron multiplication effects (for example, as described in U.S. Pat. No. 6,197,503). In other embodiments of the disclosed methods and devices, light sources, such as light-emitting diodes (LEDs) and/or semiconductor lasers may be incorporated into semiconductor chips (for example, as described in U.S. Pat. No. 6,197,503). Diffractive optical elements that shape a laser or diode light beam may also be integrated into a chip.

In certain embodiments of the present invention, a light source produces electromagnetic radiation that excites a photo-sensitive label, such as fluorescein, attached to a nucleic acid. In some embodiments, an air-cooled argon laser at 488 nm excites fluorescein-labeled nucleic acid molecules. Emitted light may be collected by a collection optics system comprising an optical fiber, a lens, an imaging spectrometer, and a 0° C. thermoelectrically-cooled CCD camera or a liquid nitrogen cooled CCD camera.

Information Processing and Control System and Data Analysis

The sequencing apparatus may comprise an information processing and control system. The embodiments are not limiting for the type of information processing and control system used. An exemplary information processing and control system may incorporate a computer comprising a bus for communicating information and a processor for processing information. In one embodiment of the disclosed methods and devices, the processor is selected from the Pentium® family of processors, including without limitation the Pentium® II family, the Pentium® III family and the Pentium® 4 family of processors available from Intel Corp. (Santa Clara, Calif.). In alternative embodiments of the disclosed methods and devices, the processor may be a Celeron®, an Itanium®, a Pentium Xeon®® or an X-scale processor (Intel Corp., Santa Clara, Calif.). In various other embodiments of the disclosed methods and devices, the processor may be based on Intel® architecture, such as Intel® IA-32 or Intel® IA-64 architecture. Alternatively, other processors may be used and the selection of processor type is elective.

It is appreciated that a differently equipped information processing and control system than the example described herein may be used for certain implementations. Therefore, the configuration of the system may vary in different embodiments of the disclosed methods and devices. It should also be noted that, while processes may be performed under the control of a programmed processor, in alternative embodiments, the processes may be fully or partially implemented by any programmable or hardcoded logic, such as field programmable gate arrays (FPGAs), TTL logic, or application specific integrated circuits (ASICs), for example. Additionally, the method may be performed by any combination of programmed general purpose computer components and/or custom hardware components.

In certain embodiments of the present invention, custom designed software packages may be used to analyze the data obtained from the detection unit 107. In alternative embodiments, data analysis may be performed, using an information processing and control system and publicly available software packages. Non-limiting examples of available software for DNA sequence 210 analysis include the PRISM™ DNA Sequencing Analysis Software (Applied Biosystems, Foster City, Calif.), the Sequencher™ package (Gene Codes, Ann Arbor, Mich.), and a variety of software packages available through the National Biotechnology Information.

### EXAMPLE 1

### Single Molecule Isolation

### Sample Preparation for DNA Sequencing

The following is a description of the techniques used to generate the samples shown in the digital photographs of FIG. 3.

Substrate Modification

A glass surface is treated with alkaline solution (NaOH, 1 N) to expose hydroxyl groups. The hydroxylated surface is subsequently treated with an aldehyde-containing silane reagent (10 millimolar in 95% ethanol) to provide an aldehyde-activated substrate. After washing with ethanol three times, and deionized water three times, the aldehyde-activated substrate is coated with a solution containing avidin and BSA (bovine serum albumin) in certain molar ratio: 1:10 or 1:1000, etc. The aldehydes react readily with primary amines on the proteins to form Schiff s base linkages between the aldehydes and the proteins, to covalently attach the proteins to the aldehyde-activated substrate surface.

Target Molecule Preparation

A DNA sample is digested with two different restriction enzymes to create DNA fragments having two different ends (for example, 10 micrograms of yeast DNA is digested in 100 microliters of 1× restriction enzyme digestion buffer (New England Biolabs), containing 50 units of EcoRI and 50 units of BamH1). About 10 nanograms of a 20 kbp DNA fragment are isolated from agarose gel by methods known by those of ordinary skill in the art. A hairpin-like oligonucleotide (cap-oligo) with a biotin moiety in the middle and a restriction enzyme site at its end is synthesized and ligated to the desired end determined by the restriction enzyme. After ligation, the DNA has a closed end with a biotin and an open end.

50 microliters of an enzyme solution containing terminal transferase (20 units) and 10 micromolar dATP can be used to add a biotinylated oligonucleotide tail (20-50 nucleotides long) to the open end of the DNA. Other end modification methods can also be used, depending on the final application of the molecule.

Beads for Attachment and Confirmation

Streptavidin coated micro-sphere (fluorescent) of 1 μm can be purchased from a commercial source (Polysciences Inc.).

Microfluidic Chip Fabrication

Designs of the micro fluidic channels to be fabricated were drawn to scale using CAD software. The designs were then printed onto transparencies using a high-resolution printer. The channels were about 100 μm in width and 2-3 cm in length. Photoresist on Silicon masters for micromolding were prepared by standard photolithography using the transparency masks and SU-8 photoresist. These patterned masters were then silanized and used for micromolding with poly (dimethyl siloxane) (PDMS). PDMS precursor was poured onto the silanized master and then cured. The cured PDMS containing the channel structure was then bonded to the modified substrate by applying pressure to enclose the channels.

Single Molecule Isolation

The modified target DNA with biotinylated ends was immobilized on the avidin/BSA substrate within the microfluidic channel by pumping a 10 nM of the target DNA solution through the microfluidic channel for 5 min using vacuum and incubating the solution for an hour. The channel was then washed with 1×PBS 3-5 times to remove any unbound target DNA. Confirmation of the attachment of the target DNA and isolation was performed by flowing a solution of 1 μm fluorescent streptavidin-coated polystyrene beads (PS) obtained from Polysciences, Inc. and observing the Brownian motion of the beads attached to the target DNA immobilized on the substrate within the microfluidic channel using fluorescent video microscopy.

The foregoing detailed description of the preferred embodiments of the disclosed methods and devices has been given for clearness of understanding only, and no unnecessary limitations should be understood therefrom, as modifications will be obvious to those skilled in the art. Variations of the disclosed methods and devices as hereinbefore set forth can be made without departing from the scope thereof, and, therefore, only such limitations should be imposed as are indicated by the appended claims.

1-32. (canceled)

33. A method for sequencing a target nucleic acid molecule comprising:
isolating a target nucleic acid molecule in a micro area on a surface of a solid substrate, wherein the solid substrate comprises a surface having at least one micro area that is coated with an amount of a specific binding agent admixed with a functional non-binding agent such that an average distance between effective binding sites allows for the creation of a micro area having a single target molecule attached to a functional binding agent,
placing the isolated target nucleic acid molecule into a reaction chamber of a microfluidic device,
sequentially digesting the monomers of the target nucleic acid from a free terminus of the target nucleic acid,
conveying the digested monomers into a detection cell operably coupled to the microfluidic device, and
sequentially detecting the digested monomers from the target nucleic acid.

34. The method according to claim 33 wherein the detection cell contains a surface coated with gold or silver and detection occurs through Raman spectroscopy.

35. The method according to claim 33 wherein the substrate is characterized by a plurality of micro areas containing isolated target nucleic acids.

36. The method according to claim 33 wherein the digestion of the target nucleic acid occurs by flowing a solution containing an exonuclease into the reaction chamber of the microfluidic device.

37. The method according to claim 33 wherein the target nucleic acid is a deoxyribonucleic acid molecule.

38. The method of claim 33 wherein the target nucleic acid is comprised of one or more monomers that are labeled with a detectable label.

39. The method of claim 38 wherein the label is detectable by fluorescence spectroscopy.

40. The method of claim 33 wherein the specific binding agent is gold and the functional non-binding agent is copper, silicon, gallium, or a combination thereof.

41. The method of claim 33 wherein the specific binding agent is avidin, streptavidin, or an antibody and the functional non-binding agent is bovine serum albumin.

42. The method of claim 33 wherein the microarea is from about 400 nm² to about 100 mm².
